(19) United States
(12) Patent Application Publication
(10) Pub. No.: US 2005/0010202 A1
(43) Pub. Date: Jan. 13, 2005

(54) Applicator for creating linear lesions for the treatment of atrial fibrillation

(75) Inventors: Vaclav O. Podany, New Fairfield, CT (US); Ebonia Washington, Basking Ridge, NJ (US); Vincenza Zaddem, Easton, PA (US)

Correspondence Address: SCULLY SCOTT MURPHY & PRESSER, PC, 400 GARDEN CITY PLAZA, GARDEN CITY, NY, 11530, US

(73) Assignee: Ethicon, Inc., Somerville, NJ (US)

(21) Appl. No.: 10/675,891

(22) Filed: Sep. 30, 2003

Related U.S. Application Data
(63) Continuation-in-part of application No. 10/609,692, filed on Jun. 30, 2003.

(51) Int. Cl. A61N 1/00; A61N 7/00

(52) U.S. Cl. 606/27; 607/4

(57) ABSTRACT

An applicator for creating a lesion in tissue, comprising one or more rigid or semi-rigid support members, a compliant material coupled to said support members, at least one passage in communication with the compliant material for infusing a medium to the compliant material and at least one electrode for conducting energy to the tissue. Further, the compliant material or other mechanical linkage may function as means for varying the distance between an ultrasonic transducer element or other ablative energy source and a surface of the tissue.

### CROSS-REFERENCE TO RELATED APPLICATIONS

This Application is a continuation-in-part of U.S. patent application Ser. No. 10/609,692, filed 30 Jun. 2003, now pending, the complete disclosure of which is hereby incorporated by reference for all purposes.

### BACKGROUND OF THE INVENTION

1. Field of Invention

The invention relates generally to the field of surgical instrumentation, and more particularly, to an applicator for creating linear lesions in living tissue.

2. Description of Related Art

Atrial fibrillation is the most common form of cardiac arrhythmia (irregular heartbeat). Irregular heartbeats are caused by abnormal electrical activity of the heart. In atrial fibrillation, the atria, the upper chambers of the heart, beat irregularly and rapidly. The erratic electrical signals may also cause ventricles, the lower chambers of the heart, to beat irregularly and rapidly. This can affect blood flow to the heart muscle and to the rest of the body.

Treatment for atrial fibrillation includes medication, or cardioversion, electrical stimulation of the heart, to restore normal sinus rhythm. Patients who do not respond to these treatments may be indicated for surgery, including catheter ablation, or more recently developed MAZE techniques.

In a traditional MAZE procedure, incisions are made in a predetermined pattern in the inter wall of the atria, which are then sutured together. Scar tissue that forms at the incisions inhibits the conduction of electrical impulses in the heart tissue that causes the fibrillation. The electrical impulses are directed along, rather than across, the incisions in a maze-like fashion that leads them to the lower ventricles of the heart.

Although generally effective, the procedure implicates the risks associated with major heart surgery. The procedure generally takes several hours, during which time the patient must receive cardiopulmonary life support. Even if successful, the procedure is highly invasive and traumatic, with full recovery taking up to six months. Additionally, the procedure requires exacting skill on the part of the surgeon.

### BRIEF SUMMARY OF THE INVENTION

Therefore, an apparatus able to produce lesions of scar tissue in the wall of the heart muscle quickly, reliably, and while minimizing damage to tissue surrounding the lesions would be highly desirable.

Provided by the present invention is an applicator for creating a lesion in tissue, comprising one or more rigid or semi-rigid support members, a compliant material coupled to said support members, at least one passage in communication with the compliant material for infusing a medium to the compliant material and at least one electrode for conducting energy to the tissue. Further, the compliant material or other mechanical linkage may function as means for varying the distance between an ultrasonic transducer element or other ablative energy source and a surface of the tissue.

At least one mechanism disclosed herein has the advantage of atraumatically clamping the tissue layer. Consistent loading can help control audible popping due to steam generation during tissue heating. The mechanisms also adapt to a variety of tissue thickness and/or local variations in thickness. Further, the mechanisms offer means to control electrode temperature elevation, avoiding tissue avulsion due to vaporization ablation. Rapid heating of the electrodes can also result in a rapid impedance change and subsequent thrombosis formation.

### BRIEF DESCRIPTION OF THE DRAWINGS

These and other features, benefits and advantages of the present invention will become apparent with reference to the following specification and accompanying drawing, in which like reference numerals indicate like features across the several views.

FIG. 1 illustrates an ultrasonic applicator according to a first embodiment of the present invention;

FIG. 2 illustrates a cross-section of the transducer head of the ultrasonic applicator, taken along the section line 2-2 of FIG. 1;

FIG. 3 is a schematic illustration of a system for creating linear lesions according to the present invention;

FIGS. 4A through 4G illustrate various embodiments of transducer heads operative to mechanically alter the depth of focus of the ultrasonic energy;

FIG. 5 illustrates a clamping mechanism according to the present invention;

FIG. 6 illustrates a further embodiment of the clamping mechanism of FIG. 5;

FIGS. 7A and 7B illustrates another embodiment of a clamping mechanism, in flaccid and turgid states; and

FIG. 8 illustrates a single-jaw embodiment having a compliant material.

### DETAILED DESCRIPTION OF THE INVENTION

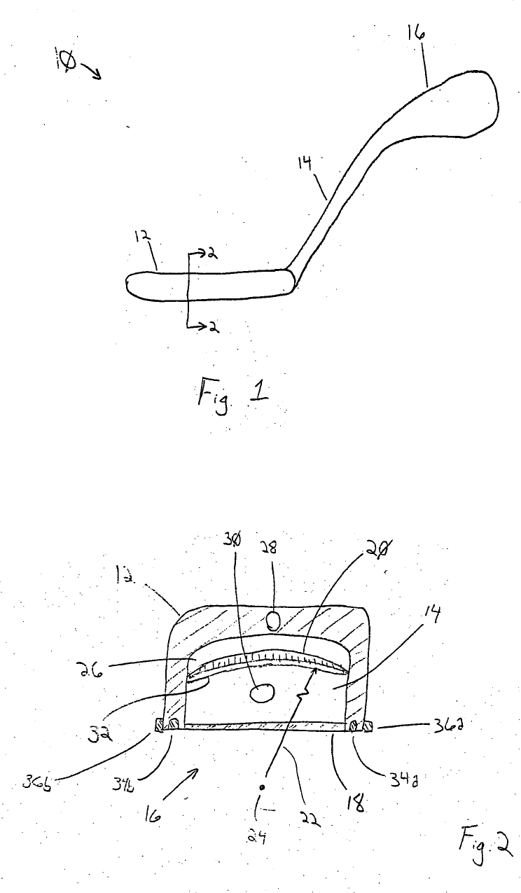

Referring now to FIG. 1, illustrates an ultrasonic applicator, generally 10, according to the present invention. Ultrasonic applicator 10 has a transducer head 12, a shaft 14, and a handle 16, by which the applicator 10 may be manipulated. Not shown in FIG. 1 are passages and cables by which power and cooling medium, respectively, are supplied to the applicator 10. These passages and cables may be either internal or external to the shaft 14.

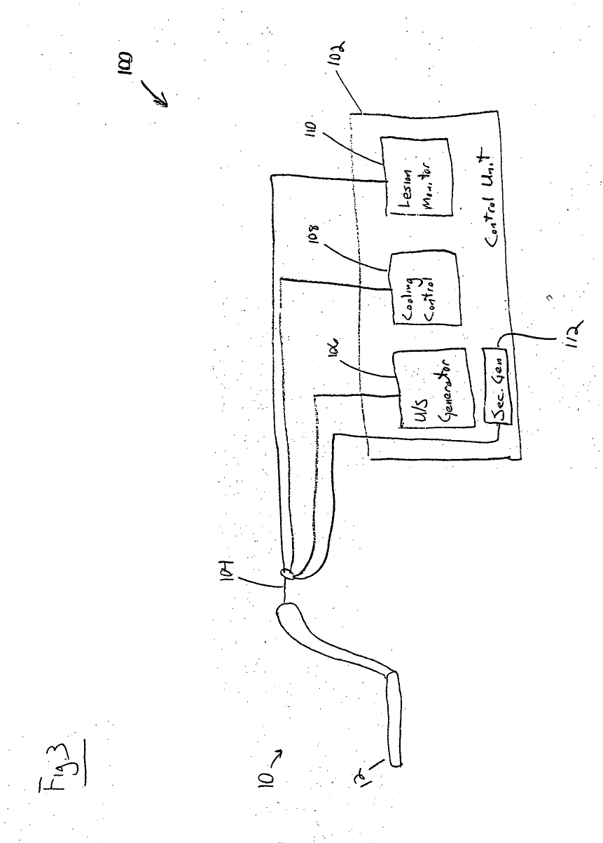

Turning now to FIG. 2, a cross section of transducer head 12 is shown, taken along line 2-2 of FIG. 1. Transducer head 12 is formed with a cavity 14 therein which is open to an acoustic window 16. The cavity 14 is sealed across the acoustic window 16 by a membrane 18. Membrane 18 is selected to have a low acoustic impedance and low coefficient of acoustic absorption, for acoustic transparency, such as films of Ultem, PET, or Styron. In one embodiment, 0.001″ thickness PEEK was used.

Located within cavity 14 is the ultrasonic vibratory element, in this embodiment a piezoelectric crystal 20. The precise piezoelectric material may be selected from among those known in the art to suit the particular application by minimizing dielectric and motional losses and inefficiencies. Further, the selected crystal may be aged, which is a logarithmic depolarization over time. A suitable aging period can reduce noticeable changes in activity.

In mounting the crystal 20, it is desirable to use a compliant mount with minimal damping. For example, epoxy on the back plate and crystal may reduce overall efficiency. In one embodiment, an RTV silicone sealant is used to mount the crystal. It is further desirable to minimize the contact area with the crystal in mounting to reduce crystal loading and heating in the mount. Particularly, elastomers absorb energy thereby reducing overall efficiency.

Piezoelectric crystal 20 has a curvature illustrated by radius 22 and converges at a focus 24 located in the direction of the acoustic window 16. The focal length may be varied, and was set to 0.25″ in one exemplary embodiment. Alternately, the transducer head 14 may be provided with a plurality of vibratory elements, either curved or flat, which form some angle with respect to one another. In either case, the ultrasonic energy will converge at some focus.

Provided in a direction opposite the focal point 24 and acoustic window 16 and adjacent the crystal 20 is an air gap 26. The air gap 26 acts as an acoustic mirror to reflect all acoustic energy from the adjacent side of the crystal 20 downward towards the acoustic window 16.

Also provided in the transducer head 12 are cooling passages 28 and 30. These cooling passages 28, 30 allow for the supply and removal of cooling medium to and from the transducer head 12. The cooling medium can include, but is not limited to, degassed water or saline. The cooling medium also provides a coupling path for the ultrasonic energy. The flow of cooling medium is determined primarily by the energy losses in crystal 20. In addition to protecting the physical integrity of the crystal, proper cooling can also minimize frequency drift in the crystal, which could otherwise cause inefficiencies.

In order to further enhance efficiency, piezoelectric crystal 20 may be provided with an impedance matching coating 32 on the side of the crystal 20 that faces the acoustic window 16. The coating 32 is shown in exaggerated thickness for illustration, and is typically on the order of one-quarter (¼) of the wavelength of the ultrasonic energy provided by the crystal 20. The selection of material and its impedance will be well known to those skilled in the art, and need not be explained further. The presence of the coating 32 impacts the cooling needs of the transducer 12, and adjustment of the coolant flow, in light of the driving power of the crystal 20, may be necessary.

Provided on either side of the acoustic window 16 are regions of porous material, 34a, 34b. This porous material 34a, 34b may be saturated with an ink, so that as the ultrasonic applicator 10 is used to form lesions in the tissue, the area where lesions have been formed will be marked by the ink. Also provided on either side of the acoustic window 16 are electrodes 36a, 36b. The electrodes 36a, 36b, may be used for pacing, i.e., electrically testing of the effectiveness of the lesions formed in inhibiting the propagation of electrical impulses through the tissue.

Alternately or additionally, electrodes 36a, 36b may be used to provide RF energy to the tissue to enhance the lesions formed by the ultrasonic energy of crystal 20. In combination with ultrasound, the RF energy can be used to form a more complete barrier or transmurality in a wider range of tissue thicknesses. This procedure is explained in more detail in U.S. patent application Ser. No. 10/609,694 (attorney Docket No. 16339) entitled Multi-Modality Ablation Device, filed 30 Jun. 2003, the complete disclosure of which is hereby incorporated by reference for all purposes. Electrodes 36a, 36b may also be adapted to transmit and/or receive ultrasound microwave, cryoablation, radio-frequency (RF), photodynamic, laser, or cautery energy, as will be discussed further, infra.

The combination of ultrasound and RF energy comprises one means for controlling the depth of the lesion in the tissue. Other means can be mechanical, for example by adjusting the focal length of the applicator. In one embodiment, the ultrasonic applicator has two crystals arranged within the transducer. By altering either or both of the angle and the distance between the two crystals, the depth of focus is adjusted. This aspect is explained further in U.S. patent application Ser. No. 10/609,693 (attorney Docket No. 16335) entitled Ultrasonic Radial Focused Transducer for Pulmonary Vein Ablation, filed 30 Jun. 2003, the complete disclosure of which is hereby incorporated by reference for all purposes.

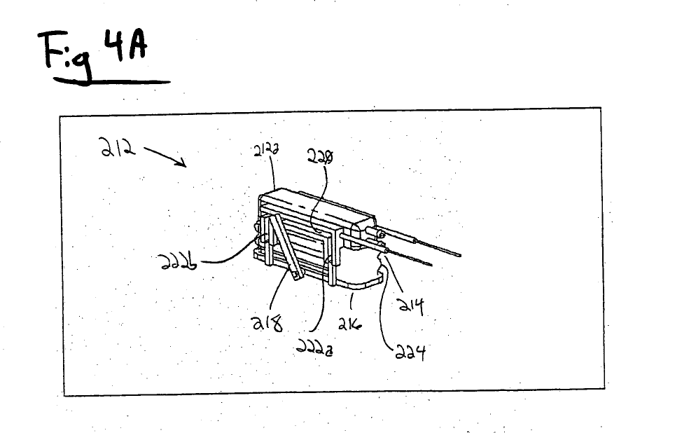

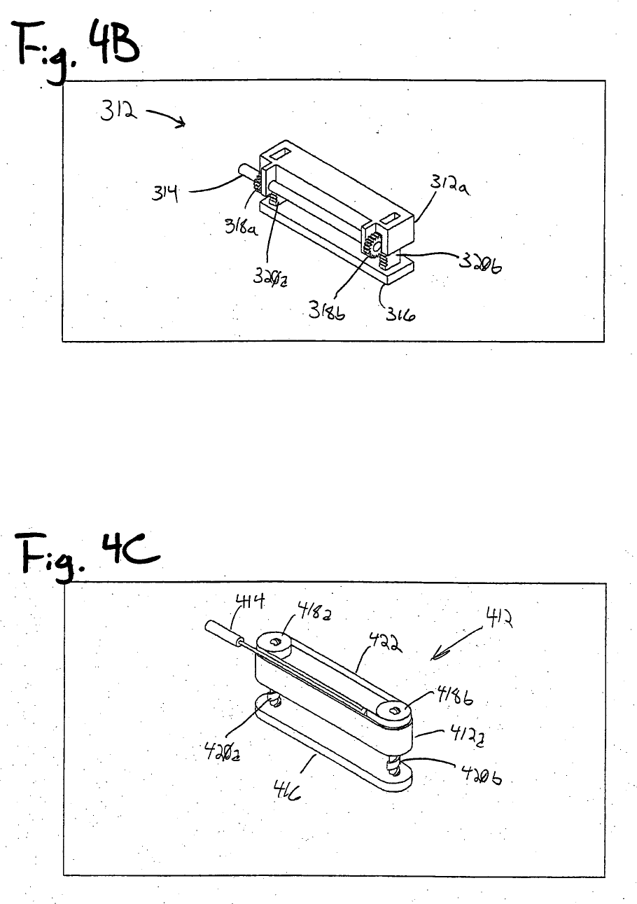

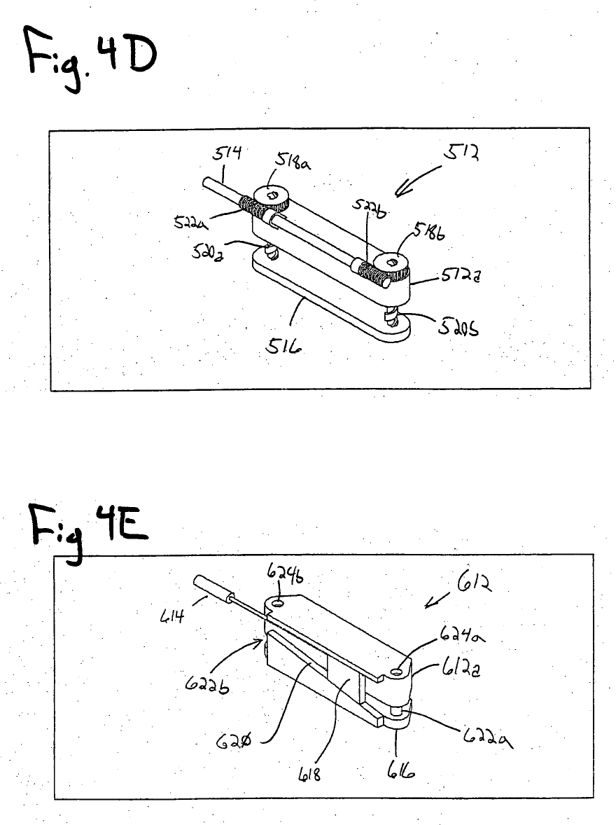

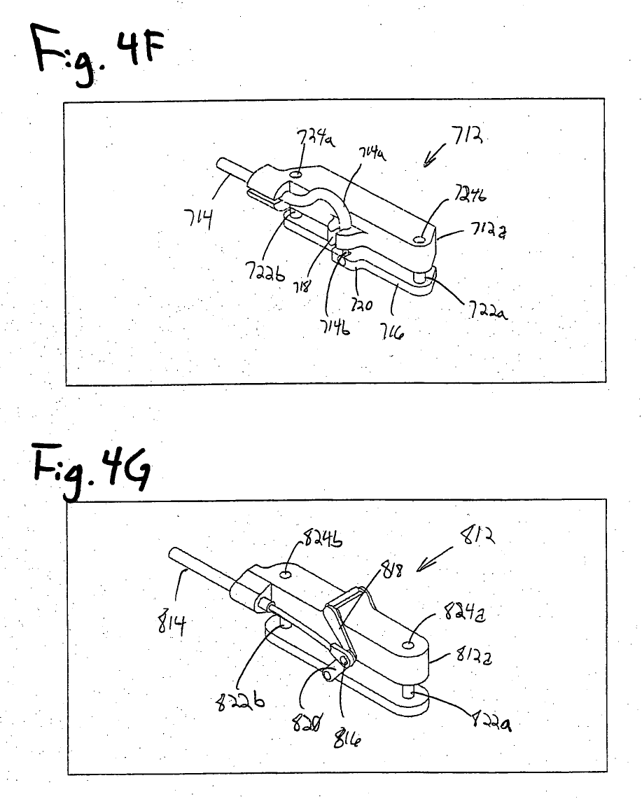

Alternately or additionally, the standoff distance between the crystal and the tissue, or between the crystal and the acoustic window, may be adjusted by mechanical means, some of which are illustrated in FIGS. 4A through 4G.

Referring to FIG. 4A, an alternate embodiment of a transducer head, generally 212, is shown. An articulating cable 214 is operatively connected to and longitudinally articulates one end of linkage 218 within slot 220. Thereby, an upper portion 212a of the transducer head 212 moves vertically with respect to the acoustic window 216, guided by slots 222a, 222b. Optionally, the space between upper portion 212a and acoustic window 216 may be enclosed by a flexible skirt 224. A similar cable/linkage/slot arrangement may be provided on an opposite side of transducer head 212.

Referring to FIG. 4B, an alternate embodiment of a transducer head, generally 312, is shown. In this embodiment, an articulating cable 314 is operatively connected to pinions 318a, 318b. The articulating cable 312 rotates the pinions 318a, 318b, which are engaged with racks 320a, 320b, respectively. Upper portion 312a of transducer head 312 is thereby moved vertically with respect to acoustic window 316.

Referring to FIG. 4C, an alternate embodiment of a transducer head, generally 412, is shown. In this embodiment, an articulating cable 414 is operatively connected to band 422. The articulating cable 412 articulates band 422, which is engaged with and synchronously rotates nuts 418a, 418b. Nuts 418a, 418b are engaged with screws 420a, 420b, respectively. As nuts 418a, 418b rotate, upper portion 412a of transducer head 412 is moved vertically with respect to acoustic window 416.

Referring to FIG. 4D, an alternate embodiment of a transducer head, generally 512, is shown. In this embodiment, an articulating cable 514 is operatively connected to worm gears 522a, 5228b. The articulating cable 512 rotates the worm gears 522a, 5228b, which are engaged with nuts 518a, 518b. Nuts 518a, 518b are engaged with screws 520a, 520b, respectively. As nuts 518a, 518b rotate, upper portion 512a of transducer head 512 is moved vertically with respect to acoustic window 516.

Referring to FIG. 4E, an alternate embodiment of a transducer head, generally 612, is shown. An articulating cable 614 is operatively connected to and longitudinally articulates a wedge 618. Acoustic window 616 may be angled, or may be provided with a sloped flange 620. A track, a sidewall, a flange, a spring or other similar device may be provided to constrain the movement of wedge 618. As wedge 618 moved against flange 618, an upper portion 612a of transducer head 612 moved vertically with respect to acoustic window 616. Upper portion 612a is guided by posts 622a, 622b, and corresponding bores 624a, 624b, respectively. A similar cable/wedge/slope arrangement may be provided on an opposite side of transducer head 612.

Referring to FIG. 4F, an alternate embodiment of a transducer head, generally 712, is shown. A sheathed cable 714 is operatively connected to transducer head 712. The sheath 714a is connected to an upper portion 712a of the transducer head 712 at bracket 718. The core 714b is connected to the acoustic window 716 at flange 720. As the core 714b moves within the sheath 714a, the upper portion 712a moves vertically with respect to the acoustic window 716. This motion is guided by posts 722a, 722b, and corresponding bores 724a, 724b, respectively. A similar cable/flange arrangement may be provided on an opposite side of transducer head 712.

Referring to FIG. 4G, an alternate embodiment of a transducer head, generally 812, is shown. An articulating cable 814 is operatively connected to transducer head 812, and to a common pin 816 joining linkages 818 and 820. As pin 816 moves longitudinally, the upper portion 812a moves vertically with respect to the acoustic window 816. This motion is guided by posts 822a, 822b, and corresponding bores 824a, 824b, respectively. A similar cable/linkage arrangement may be provided on an opposite side of transducer head 812.

Further, selection of the frequency of the ultrasonic wave can be used to control the depth and transmurality of the lesion. Lower frequencies are less absorbed by the tissue and provide deeper penetration. The higher frequencies have higher absorption in the tissue and this provides higher rate of heating but lower penetration. Therefore, by selecting or optimizing the frequency of the crystal 20, the depth of penetration of the ultrasonic energy and the heating rate can be adjusted so that a range of tissue thickness can be ablated, thereby controlling the depth of the lesion. A predetermined target may be established based upon the thickness of the tissue, or a thicker lesion may be formed by adjusting the frequency in process. Control of the ultrasonic frequency comprises yet another means for controlling the depth of the lesion.

Alternately or additionally, either or both of electrodes 36a, 36b, can be made responsive to ultrasound. These can then be used to receive a lower power inspection ultrasound signal, emitted after the lesion is formed to inspect the physical properties of the lesion.

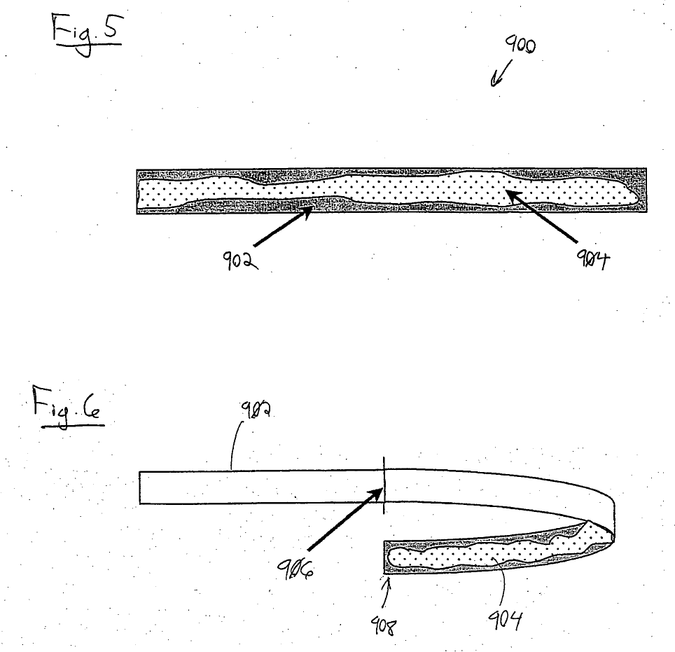

Referring now to FIG. 5, shown is a clamping mechanism which can be adapted to the present invention. The clamping mechanism, generally 900, includes a rigid or semi-rigid member 902 coupled to a compliant material 904. The compliant material can include, but is not limited to, polyethylene terephthalate (PET), flexible polyvinyl chloride (PVC), nylon, polyolefin, polyurethane, latex, silicone, or other elastomers knows to be used in the manufacture of expansible members, used for example for fixation and/or occlusion. The compliant material 904 can transition between a flaccid and turgid states by the infusion of a medium, for example, fluid, gas, gel, rheological material, or other media which affects turgidity. The medium may also consist of a combination of materials, such as a slurry of solid particles suspended in a solvent gas or liquid. In that case, removing the solvent would transition the compliant material 904 from a turgid state to a rigid state.

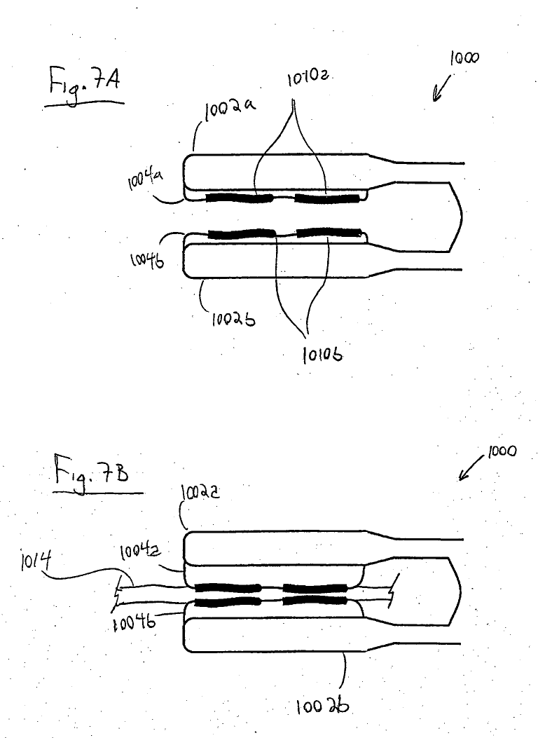

Referring now to FIG. 6, the clamping mechanism 900 is shown with the rigid member partially formed into the shape of a “P”. A latching mechanism 906 can be provided to hold the distal end 908 in place proximally. Placing the clamping mechanism 900 around tissue to be clamped, by distending the compliant material 904 the tissue is compressed atraumatically. This particular arrangement is particularly well suited for application to hollow tissue structures, for example atrial chambers or pulmonary veins.

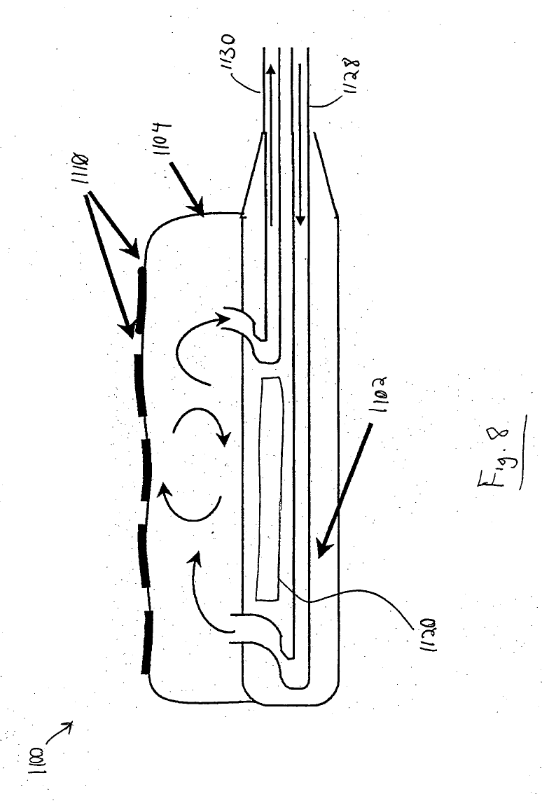

Referring now to FIG. 7A, shown is another embodiment of a clamping mechanism, generally 1000. In this embodiment, clamping mechanism 1000 has rigid or semi-rigid jaws 1002a and 1002b, respectively. The jaws as shown are fixed relative to one another, but may articulate in a further embodiment. In this embodiment both jaws are provided with a compliant material 1004a, 1004b, but either one may have a compliant material without the other.

Also shown are electrodes 1010a and 1010b, which may be provided on the surface of the compliant material 1004a and 1004b, respectively. Electrodes 1010a and 1010b may consist of a conductive material or an array of conductive surfaces in any geometry. Alternately or additionally, they may comprise conductive elements integrated into the surface of the compliant material. For example, a fiber of carbon or another material conductive of electricity, RF or whichever type of energy the electrode is to be responsive to, may be woven into a bounding surface of compliant material 1004a, 1004b. The electrodes 1010a and 1010b are operatively connected to an energy source, for example ultrasound, microwave, cryoablation, radio-frequency (RF), photodynamic, laser, or cautery. The four (4) electrodes shown are merely exemplary, and their number may be more or less.

Alternately or additionally, an ultrasonic vibratory element may be provided in one or both of jaws 1002a, 1002b. Additionally, a reflector may be provided with either or both of jaws 1002a, 1002b to reflect and/or focus incident energy.

Referring now to FIG. 7B, clamping mechanism 1000 is illustrated having compliant material 1004a, 1004b in a turgid state. The turgid compliant material 1004a, 1004b is shown compressing and thereby securing a tissue layer 1014. Once secured, energy can be applied to the electrodes and/or transducer to ablate the tissue and form the desired lesions therein.

Referring now to FIG. 8, shown is a single jaw embodiment 1100. The single jaw has a rigid or semi-rigid member 1102, and a compliant material 1104, in this case shown in a distended or turgid state. A plurality of electrodes 1110 are shown on the surface of the compliant material 1104. Also shown are passages 1128 and 1130, which provide for the inflow and outflow of the a medium for altering the turgidity of the compliant material 1104. In this or other embodiments described above, the surface of the compliant material may be textured to reduce tissue slipping. In this or other embodiments described above, the turgidity inducing medium may be circulated to serve a heat sink for the ablation process.

Provided in this embodiment is an ultrasonic vibratory element 1120. In operation, the infusion of a turgidity inducing medium can alter the distance between the ultrasonic vibratory element 1120 and the tissue surface, thereby varying the depth of focus and penetration of the ultrasonic energy. Therefore, embodiments including conforming material as described above will be seen as yet another means of controlling the depth of lesion formed in the tissue.

Referring now to FIG. 3, the system, generally 100, for creating linear lesions according to the present invention is shown. The ultrasonic applicator 10 is connected to control unit 102 via a conduit 104. Conduit 104 provides the pathways necessary for electrical, RF, and/or fluid communication with the transducer head 12.

Control unit 102 comprises a ultrasonic generator 106, which supplies power of the appropriate frequency to the crystal 20 for the production of acoustic energy. It would be desirable to provide compensation for the static capacitance of the crystal 20 in order to reduce the capacitive load on the ultrasonic generator 106. It would also be desirable to match the impedance of the crystal 20 to the ultrasonic generator 106 to minimize reflections from the load. Also, where wire and solder joints are used to connect the crystal 20 to the ultrasonic generator 106, it would be desirable to use a light wire and small solder joints at the crystal interface. Additional mass of these items can alter the frequency of the crystal. Further, proper solder technique can have an impact, because excess heat caused by poor solder joints can depolarize a ceramic crystal.

Control unit 102 also provides a coolant control section 108. Coolant control section 108 can include a pump for the circulation of cooling medium, sensors for monitoring the temperature of the coolant fluid, and in closed cooling systems, a heat exchanger for expelling heat from the coolant fluid before it is recycled back into the transducer.

Control unit 102 also comprises a lesion monitoring section 110. In combination with electrodes 36a, 36b, once formed, the lesions created can be tested for effectiveness by electrical pacing, discussed supra, or by monitoring the tissue impedance. Additionally or alternately, other methods of monitoring the effectiveness the lesions, including but not limited to, ultrasound imaging, can be employed to verify the suitability of the lesions formed. Additionally, the control unit may comprise a secondary generator 112, for applying ultrasound, microwave, cryoablation, radio-frequency (RF), photodynamic, laser, or cautery energy to the tissue at the transducer 12, as discussed, supra.

The operation of the system 100, according to the present invention will now be described. Typically, the surgeon will establish access to the epicardium through sternotomy, thoracotomy, or less invasively, by thorascopic port access. The transducer 12 is placed on the surface of the heart where the lesion is to be formed. A trigger switch, which may be located on the shaft 14 of the applicator 10, alternately embodied as a foot pedal for the surgeon, or on the control unit 102, activates the ultrasonic generator 106 to introduce ultrasonic energy to the tissue.

The ultrasonic generator 106 applies electrical energy to the crystal 20 to induce ultrasonic vibration. In one embodiment, the crystal was tuned to 8.72 Mhz and employed a power setting of 60W. In this exemplary embodiment, acoustic intensity along the focal line including focal point 24 is in a range between 1,000 and 1,500 w/cm², sufficient to coagulate tissue within a short period of time. In vitro testing indicates the transmural lesion in tissue of typical thickness can be made in about 15 to 30 seconds.

The present invention has been described herein with reference to certain exemplary embodiments. Certain modifications and alterations may be apparent to those skilled in the art without departing from the scope of the present invention. The exemplary embodiments are meant to be illustrative, and not limiting, on the scope of the invention, which is defined by the appended claims.

1. An applicator for creating a lesion in tissue, the applicator comprising:
a first rigid or semi-rigid support member;
a first compliant material coupled to said first, support member;
a first passage in communication with said first compliant material for infusing a medium to the compliant material coupled to the first support member; and
at least one electrode for conducting energy to the tissue.

2. The applicator according to claim 1, further comprising:
a second rigid or semi-rigid support member;
a second compliant material coupled to said first support member; and
a second passage in communication with said second compliant material for infusing a medium to the compliant material coupled to the second support member.

3. The applicator according to claim 2, wherein said first and second support members are fixed relative to one another.

4. The applicator according to claim 2 wherein said at least one of said first and second support members is operative to articulate relative to the other.

5. The applicator according to claim 2 wherein said first and second support members are located such that said each of the first and second compliant materials extends in the direction of the other.

6. The applicator according to claim 2, further comprising at least one reflector operative to reflect and or focus incident energy located on one of said first and second support members.

7. The applicator according to claim 1, wherein said electrode is located on the surface of said first compliant material.

8. The applicator according to claim 1, wherein said electrode is integrated into the surface of said first compliant material.

9. The applicator according to claim 1, further comprising a third passage in communication with said first compliant material, which together with said first passage provides for the circulation of said medium.

10. The applicator according to claim 2, further comprising a fourth passage in communication with said second compliant material, which together with said second passage provides for the circulation of said medium.

11. An applicator for creating a lesion in tissue, the applicator comprising:
a first rigid or semi-rigid support member,
an ultrasonic transducer element mounted to said first support member; and
means for varying the distance between the ultrasonic transducer element and a surface of the tissue.

12. The applicator according to claim 11, further comprising a first passage in communication with said compliant material for infusing a medium to said compliant material, wherein said means for varying the distance between the ultrasonic transducer and the surface of the tissue comprises a compliant material coupled to said support member.

13. The applicator according to claim 12, further comprising a second passage in communication with said compliant material, which together with said first passage provides for the circulation of said medium.

14. The applicator according to claim 12 further comprising at least one electrode for conducting energy to the tissue.

15. The applicator according to claim 14, wherein said electrode is located on the surface of said first compliant material.

16. The applicator according to claim 14, wherein said electrode is integrated into the surface of said first compliant material.

17. The applicator according to claim 11 further wherein said means for varying the distance between the ultrasonic transducer and the surface of the tissue comprises a mechanical means for varying the standoff distance.

18. The applicator according to claim 17, further comprising an actuating cable operatively connected to said mechanical means and operative to vary said standoff distance.

19. The applicator according to claim 17 further comprising at least one electrode for conducting energy to the tissue.
